(19) United States
(12) Patent Application Publication
(10) Pub. No.: US 2003/0082147 A1
(43) Pub. Date: May 1, 2003

(54) Bacteria for insect control

(75) Inventors: Dawn H. Gouge, Chandler, AZ (US); Ralph A. Dudney, Richmond, TX (US); Kirk A. Smith, Chandler, AZ (US)

Correspondence Address: Mark R. Wisner, WISNER & ASSOCIATES, Suite 930, 2925 Briarpark, Houston, TX, 77042-3728, US

(73) Assignee: Xeno Insecticides, Inc.

(21) Appl. No.: 09/941,017

(22) Filed: Aug. 28, 2001

(51) Int. Cl. C12N 1/20

(52) U.S. Cl. 424/93.3; 435/252.1; 435/253.3; 424/93.47

(57) ABSTRACT

Insecticidally-active bacterial cultures and methods of maintaining the virulence of the bacterial cultures. Several species of the bacteria are harvested from nematodes of the families Steinernematidae and Heterorhabditidae maintained in suitable insect hosts and then cultured as separate, isolated cultures under conditions suitable to the reproduction of the particular bacterial specie(s). Bacteria are harvested for use as an insecticide during the exponential phase of population growth so that the bacteria are highly motile, optionally mixed with other populations of bacteria harvested and cultured from suitable nematode hosts, diluted in water and applied to the insects to be controlled by, for instance, spraying or, in the case of such insects as ants and termites, by pouring the dilute, harvested bacteria onto the insect mound. To maintain the virulence of the isolated bacterial cultures, the bacteria are periodically re-isolated from the nematode, infected in the insect host, and then re-cultured.

### BACKGROUND OF THE INVENTION

The present invention relates generally to the use of certain types of bacteria for the control of insect pests, and more particularly, to methods for isolating and culturing symbiotic and non-symbiotic bacterial populations that are insecticidally active and methods of using such bacteria for control of insects, particularly insect pests such as termites and fire ants.

The mutualistic association between the entomopathogenic nematodes in the families Steinernematidae and Heterorhabditidae and Enterobacteriaceae bacterial species such as Xenorhabdus spp. and Photorhabdus spp. is well documented. Both nematode families act as vectors that inoculate insect hemolymph with these bacterial genera with the result that the bacteria multiply rapidly and kill the insect host within 48 hours by causing septicemia. Bacterial symbionts produce a variety of anti-microbial compounds to suppress the growth of contaminants and/or competing pathogens (Paul, V., et al., Antibiotics in microbial ecology: Isolation and structure assignment of several new antibacterial compounds from the insect-symbiotic bacteria Xenorhabdus spp., 7 J. Chem. Ecol. 589-597 (1981)) and phase variation is common. The nematodes themselves release an immune-suppressive factor (Gotz, P., et al., Interactions between insect immunity and an insect-pathogenic nematode with symbiotic bacteria, 212 Proc. Royal Soc. London Ser.B 333-350 (1981)), and the result of the rapid growth, the release of anti-microbial compounds, phase, and immune suppression is that the bacteria is quite pathological to the insect host.

Current information indicates that, in spite of the lethality of the bacteria, the bacteria are not suited for use as an effective insecticide by themselves. It is generally considered that the bacteria are unable to access insect hemolymph unless inoculated or injected into the insect body by the nematodes. Consequently, the bacteria are ineffective against insect pests without the nematodes. Further, the bacteria do not have an environmentally resistant stage such that their ability to survive without the nematodes may be questionable.

The present invention, however, is premised upon the surprising discovery that certain enterobacterial species will, both alone and in mixtures with other bacterial species and with and without the presence of an adjuvant, function as biological control agents against insect pests under certain conditions. In one aspect, therefore, it is an object of the present invention to provide bacterial species and isolates that, when utilized in accordance with the present invention, function as biological control agents against insect pests. It is also an object of the present invention to provide methods of isolating and culturing bacteria for use as an insecticide.

### SUMMARY OF THE INVENTION

These objects, and others that will be apparent to those skilled in the art who have the benefit of the disclosure set out herein, are accomplished by providing a method of maintaining the virulence of bacteria harvested from nematodes comprising the steps of maintaining a library of nematodes selected from the families Steinernematidae and Heterorhabditidae in a suitable culture media under conditions conducive to the health of the nematodes and maintaining separate populations of the bacterial species harvested from the nematodes in suitable culture media under conditions conducive to the health of the bacterial species. The separate populations of the bacterial species are periodically re-isolated from the nematode host selected from the library of nematodes and an insect host is inoculated under conditions conducive to the reproduction of the inoculated bacterial species. After several generations within the host, the bacteria is then re-isolated for separate culture.

In another aspect, the objects of the present invention are achieved by providing an insecticidally-active composition comprising a mixture of one or more bacterial species harvested from nematodes of the families Steinernematidae or Heterorhabditidae, the nematodes being cultured in a suitable insect host, and stored in water. The bacterial species comprising the composition is selected from the genera Xenorhabdus, Photorhabdus, Pseudomonas, Burkholderia, Flavobacterium, Enterobacter, Serratia, Xanthomonas, and Vibrio.

### BRIEF DESCRIPTION OF THE DRAWINGS

FIGS. 1 and 2 are graphs of the growth curves of Xenorhabdus nematophilus (Im-1) in culture measured in optical density and per cent motility for a static (FIG. 1) and shaken culture (FIG. 2).

FIGS. 3 and 4 are bar graphs showing per cent mortality of first and second experiments with ants as a function of time after exposure to several Xenorhabdus and Photorhabdus species as listed infra.

FIG. 5 is a bar graph similar to FIGS. 3 and 4 showing per cent mortality of termites.

### DESCRIPTION OF THE PREFERRED EMBODIMENT

It is well known that most, if not all, bacteria gradually lose their virulence when repeatedly cultured in artificial media, and this loss of virulence is one reason the symbiotic Enterobacteria may not be effective as an insecticide without the nematode vector. The present invention is premised in part on the surprising discovery that the nematode vector can itself be used to maintain virulence. Specifically, bacterial virulence is maintained by simply passing the bacterium through a suitable (axenic) nematode vector selected from an in vivo bank of desired nematode strains and then “re-isolating” the bacterial species/strain from infected insect hemocoel.

A library of nematodes selected from the families Steinernematidae and/or Heterorhabitidae is maintained in suitable culture medium under conditions conducive to the health of the nematodes. Separate populations of bacterial species harvested from nematodes are maintained in suitable culture medium under conditions conducive to the growth of the culture. Examples of conditions under which the nematode and bacterial populations are maintained are described in more detail below, but suitable methods for culturing nematodes are also described in such known sources as Woodring, J. L. et al., (Steinernematid and heterorhabditid nematodes: A handbook of techniques (Southern Cooperative Series Bulletin 331) and bacteria may be cultured, for instance, on soy agar plates using conventional techniques. A bacterium selected from the separate bacterial cultures is periodically inoculated into axenized nematodes selected from the separate library of nematodes under conditions that are conducive to the reproduction of the inoculated bacterial species. After one or more generations in an insect host, the bacterium is then re-isolated for separate culture.

In practicing this method, it has been found that the results are often variable as far as restoring bacterial virility is concerned. However, virility need only be restored to one culture to provide the desired result.

The present invention is also premised in part on the culturing of the bacteria “on site,” e.g., at a location proximate the location at which the bacteria is to be used for insect control. Although not a requirement of the present invention, for reasons to be made clear below, on site culturing of the bacteria is advantageous. On-site culturing of the bacteria is also advantageous at the initial commercial production stage since only minimal volumes of bacteria are required for use in inoculating the on site cultures and suitable quantities of bacteria are packaged for shipping as described in our prior U.S. Pat. No. 5,733,777, which patent is hereby incorporated into this specification in its entirety by this reference thereto. Packaging is accomplished with sterile fermentation equipment and the culture is reduced in volume to, for instance, 10% of its original volume and that volume is optionally further reduced by placing the inoculum onto sterile filter paper that is air dried or freeze dried to be used as inoculum for on site culture. The sealed media bottle has a shelf life of from about 2 to about 5 years. The inoculum disks used in the second cap of the media bottle disclosed in that patent also have a long shelf life, but even if shelf life is exceeded, only the inoculum disk need be replaced and the media, which is relatively expensive, is not lost.

Preparation of the filter disk is by several ways known in the art, but in a presently preferred method, an 18-48 hour bacterial culture is centrifuged to concentrate the bacterial cells and the cells are then re-suspended in a suitable medium. In a presently preferred embodiment, the medium is a mixture of LB broth (Miller) (1% Tryptone (Difco), 0.5% yeast extract (Difco), 1% NaCl) and skim milk in a 1:10 ratio. One or two drops of the resulting mixture is pipetted onto a sterile filter paper disk sized to fit the above-described media bottle and air dried at 25-30° C. for packaging.

As noted above, so far as was previously known in the art, the bacteria were not capable of infecting the insect hemolymph without the nematode or by injection into individual insects. In a third aspect, therefore, the present invention is premised on the discovery that if the insects are inoculated during the time the bacteria are highly motile, and provided with enough liquid to allow the bacteria to move, the bacteria can enter the insect bodies. It has been found that the period of peak motility is also the period of exponential growth of the culture and that the activity of the culture can be scanned with a simple test to determine motility, and therefore, the insecticidal activity of the culture. Under ideal conditions, this phase of the growth of the culture generally occurs after approximately 30-48 hours and the maximum level of motility is sustained for approximately another 60 hours thereafter; lower temperature and/or changes in other environmental factors affect the growth of the culture in ways generally known to those skilled in the art such that, rather than requiring that a culture be utilized as an insecticide at some specific time, the present invention contemplates that the culture will be utilized at a time when motility is relatively high. After the period of sustained motility (and high insecticidal activity), the motility of the bacteria declines at approximately the same rate as the growth.

The active culture is preferably applied to the insect by spraying, but the present invention contemplates the use of most other methods of application such as, for instance, bait or by drenching an ant mound with a liquid suspension of the culture. If it is applied as a spray or drench, the culture may be diluted for application with water or other suitable liquid(s); dilutions of about 50:1 (liquid:active culture) may be utilized to advantage, but in dry conditions, higher ratios such as 80:1 may help provide adequate moisture for motility.

Selection and culture of bacteria was accomplished as follows. Although the insect larva from Galleria mellonella L. was used to advantage in the examples described herein, those skilled in the art will recognize from this disclosure that many other host insect larvae may be used to advantage in the harvesting of bacteria of the present invention, it being preferred that the host insect larvae be selected from that group of insects that have a minimal immune response (as measured by conventional techniques known in the art) to the bacteria. It is also preferred that the larvae that are utilized be readily available at low cost and that the species grows quickly, easily, and in quantity in culture.

Regarding the species/strains of juvenile entomopathogenic nematode used to infect host insect larvae, ten species/strains of Steinernema spp. or Heterorhabditis spp. were used to advantage in the examples described herein. Other species/strains of nematode may be used to advantage for larval infection. Those skilled in the art who have the benefit of this disclosure will recognize that any number or combination of specie/strain of nematode may be used to infect the host insect larvae.

It will also be evident to those skilled in the art from this description that temperature is one of the most important factors affecting the nematode/bacterial life cycle, and that generally nematodes have greater thermal restrictions than their bacterial partners. Thus, different experimental incubation temperatures may be used to establish bacterial species favoring one temperature over another.

In the embodiments of the present invention described below, three different temperatures were used to advantage. In one set of examples, infected larvae were incubated at 22° C., 27° C. and 32° C. At set intervals, infected larvae were removed from culture. The interval used to advantage in the examples described herein was a six hour interval, but again, those skilled in the art will recognize that other intervals may be used to advantage depending upon such factors as the particular larvae utilized, temperature, culture media, and other factors that are well known. Every six hours over a 48-hour period, larvae were removed from culture, rinsed, and dorsally dissected. Hemolymph was collected with a sterile loop and streaked on tryptic soy agar plates, and allowed to grow into bacterial colonies. Individual bacterial colonies are then collected, cultured, and when possible, the bacterial species are identified.

Several experimental designs are presented below to illustrate the production of non-symbiotic bacteria in insect larvae using infective juvenile entomopathogenic nematodes as vectors and the method of harvesting bacteria from infected larval hemolymph. However, the following examples are provided for illustrative purposes only and are not intended to limit the scope of the invention.

### MATERIALS

Nematode source. Steinernema riobrave Cabanillas, Poinar and Raulston (Oscar) was obtained from Dr. James R. Raulston (USDA-ARS, Weslaco, Tex.) and was originally isolated from soil samples collected from the Lower Rio Grande Valley, Tex. S. riobrave (355), Steinernema feltiae (Filipjev) (27), and S. carpocapsae (25) were cultured from commercial samples formulated in a variety of media (Biosys Inc, Columbia, Md.). S. carpocapsae (Guardian) was cultured from a commercial sample formulated on sponge (Hydro Gardens, Colorado Springs, Colo.). S. carpocapsae (Kapow and Mexican strains) have been in laboratory culture for several years. Heterorhabditis bacteriophora Poinar (Cruiser) was cultured from a commercial sample, formulated in a clay based substrate (Ecogen, Langhorne, Pa.). Heterorhabditis marelatus Liu and Berry has been in laboratory culture for less than one year, and was isolated in Oregon. Heterorhabditis megidis Poinar, Jackson, and Klein (M-145) has been in laboratory culture for several years and originated from Ireland.

Infective juvenile (IJ) entomopathogenic nematodes were obtained by in vivo culturing (Woodring, J. L., et al., Steinernematid and heterorhabditid nematodes: A handbook of techniques (Southern Cooperative Series Bulletin 331), Fayetteville, Ark.: Arkansas Agricultural Experiment Station (1988)) in late instar Galleria mellonella L. larvae reared at 27° C. for S. riobrave and H. bacteriophora, and 22° C. for H. marelatus, H. megidis, S. carpocapsae, and S. feltiae. IJs were extracted using White traps (White, G. F., A method for obtaining infective nematode larvae from cultures, 66 Science 302-303 (1927)), and stored in distilled water at 15° C. (S. riobrave, H. megidis, and H. bacteriophora), and 7° C. (S. carpocapsae, H. marelatus, and S. feltiae S. feltiae). Nematodes were stored for no longer than 4 days prior to use.

Isolation of Bacteria from Insect Hemocoel Infected with Entomopathogenic IJs

Individual late instar G. mellonella were placed on the surface of filter paper contained in 35 mm petri dishes. IJs were acclimated at 22, 27, or 32° C. for 4 hr then pipetted onto the filter paper surface at a dose rate of 400 IJs per petri dish. Five replicate petri dishes were made for each of the 10 nematode strains/species and 3 incubation temperatures (22, 27, and 32° C.). Dishes were sealed with PARAFILM®, then incubated. At six-hour intervals over a 48-hour period, larvae were removed, rinsed 3 times in sterile distilled water, and dissected dorsally between the 5th and 6th interstitial segment. Hemolymph was collected with a sterile loop and streaked on tryptic soy agar plates.

The bacterial colonies were grown for 48 hours at 27° C. Single colonies showing morphological differences (color, shape, size) were removed using a sterile needle and transferred to fresh tryptic soy agar plates. Thereafter, pellicle and clumped colonies were collected from the pure cultures and carbon source utilization tests conducted using the BIOLOG® gram negative identification system. BIOLOG® multi-well plates were analyzed after 24 hours incubation at 27° C. Assimilation characteristics were used to identify Gram negative bacterial species.

The form variant of symbiotic bacteria identified as Xenorhabdus or Photorhabdus was determined by using the adsorption of neutral red from MacConkey agar as an indicator of primary form (Boemare, N. E., Recherches sur les complexes nemato-bacteriens entomopathogenes: Etude bacteriologique, gnotoxenique et physiopathologique du mode d'action parasitaire de Steinernema carpocapsae Weiser (Rhabitida: Steinernematidae), Ph.D. Thesis, Universite des Sciences, Montpellier, France (1988)). Bioluminescence of Photorhabdus bacterial colonies was observed in a photographic darkroom after 6 minutes of eye acclimation.

To establish which bacterial species were carried by G. mellonella, twenty uninfected larvae were dissected, and the contents of the insect guts streaked on tryptic soy agar. Bacterial colonies were identified using the BIOLOG® system.

Isolation of Bacteria Directly from IJs

Both untreated nematodes and surface sterilized nematodes were investigated. Untreated nematodes were suspended in sterile distilled water. To surface sterilize nematodes, infective juveniles were transferred to a 0.1% solution of thimerosal (merthiolate, sodium ethylmercurithiosalicylate) for 20 m, then rinsed 3 times in sterile distilled water (method modified from Akhurst, R. J., Morphological and functional dimorphism in Xenorhabdus spp., bacteria symbiotically associated with the insect pathogenic nematodes, Neoaplactana and Heterorhabditis, 121 J. Gen. Microbiol. 303-309 (1980)). Approximately 300 IJ nematodes were then pipetted under sterile conditions onto tryptic soy agar plates. The bacterial colonies were grown for 48 hours at 27° C. Single colonies showing morphological differences (color, shape, and size) were removed using a sterile needle and transferred to fresh agar. Thereafter, pellicle and clumped colonies were collected from the pure cultures and carbon source utilization tests conducted using the BIOLOG® Gram negative identification system. BIOLOG® multi-well plates were analyzed after 24 hours incubation at 27° C. Assimilation characteristics were used to identify Gram negative bacterial species.

Infective juveniles of S. feltiae (27), S. riobrave (Oscar), or Steinernema carpocapsae (Kapow) (Weiser) were prepared in 2 ways: nematodes were untreated, or surface sterilized, then pipetted under sterile conditions onto tryptic soy agar plates. The plates were incubated at 27° C. Several more species of associated bacteria were identified using this method. There was no difference in bacterial species identified from non-sterile or surface sterilized nematodes.

Three common bacterial colonies were repeatedly isolated from the gut contents of G. mellonella. The contaminants were identified as Salmonella subspecies 1 G, Pasteurella caballi, and Xanthomonas campestris PV visicatoria B. Following nematode application, several other bacterial species were identified from nematode infected insect cadavers (Table 1); these were considered to have originated from the nematodes. Several non-symbiotic bacterial species were identified from infected insect cadavers: Enterobacter gergoviae, Vibrio spp., Pseudomonas fluorescens type C, Serratia marcescens, Citrobacter freundii, and Serratia proteomaculans. At 18-24 hours incubation, the primary symbiont occurred almost exclusively. Few agar plates during this period grew any other cultures with the exception of Heterorhabditis megidis Poinar, Jackson, and Klein (M-145). The primary symbiont from H. megidis (M-145) disappeared rapidly being replaced at 24 hours with Serratia proteomaculans. Secondary bacterial associates generally appeared outside the 18-24 hour window. G. mellonella mortality usually occurred within the first 18 hours. At higher temperatures the primary symbiont appeared earlier, with the exception of symbionts from Steinernema feltiae (Filipjev) (27) and H. megidis (M-145). Two strains of nematodes carried only the primary symbiont: Steinernema riobrave Cabanillas, Poinar and Raulston (355) and

TABLE 2
Bacterial species identified from Galleria mellonella hemolymph infected with entomopathogenic nematodes at 22° C. over time

| | | | | S. | S. | S. | S. | | H. bact- | |
|---|---|---|---|---|---|---|---|---|---|---|
| | S. | S. riobrave | S. riobrave | carpocapsae | carocapsae | carpocapsae | carpocapsae | H. | eriophora | H. megidis |
| Time | feltiae (27) | (355) | (oscar) | (25) | (Kapow) | (Mexican) | (Guardian) | marelatus | (Cruiser) | (M-145) |
| 0 | Salmonella sp., Pasteurella caballi, and Xanthomonas campestris isolated from uninfected G. mellonella | | | | | | | | | |
| 6 | P. caballi | X campestris | X. camp- | P. caballi | Vibrio sp. | P. caballi | P. caballi | P. caballi | X. camp- | X. camp |
| | and Salmon- | | estris And X. | and Salmon- | and X. | | and Salmon- | | estris | estris and |
| | ella sp. | | maltophilia | ella sp. | nematophilus | | ella | | | Salmonella |
| | | | | | | | | | | sp. |
| 12 | P. caballi | X. camp- | X. camp- | P. caballi | X. | P. caballi | P. caballi | Vibrio sp. | X. camp- | X. camp- |
| | | estris | estris | | nematophilus | and Vibrio | and Vibrio | | estris | estris and |
| | | | | | and P. cab- | sp. | sp. | | | Photo- |
| | | | | | alli | | | | | rhabdus sp |
| 18 | P. caballi | X. campe- | Xenorhabdus | X. | X. | X. | X. | Photohabdus | X. camp- | Photo- |
| | and X. bo- | estris | sp. | nematophilus | nematophilus | nematophilus | nematophilus | sp. | estris | rhabdus sp. |
| | veinii | | | | | | | | | |
| 24 | X. boveinii | Xenorhabdus | Xenorhabdus | X. | X. | X. | X. | Photo- | P. lumin- | S. |
| | | sp. | sp. | nematophilus | nematophilus | nematophilus | nematophilus | rhabdus sp. | escens | proteo- |
| | | | | | | | | | | maculans |
| 30 | Vibrio | Xenorhabdus | Xenorhabdus | X. | X. | X. | X. | Photo- | P. lumin- | S. |
| | cholerae, | sp | sp. | nematophilus | nematophilus | nematophilus | nematophilus | rhabdus sp. | escens | proteomac- |
| | | | | | | | | | | ulans and Vi- |
| | | | | | | | | | | brio met- |
| | | | | | | | | | | schrikovii |
| 36 | S. mar- | Xenorhabdus | Xenorhabdus | X. | X. | X. | X. | Photo- | P. lumin- | E. gergoviae |
| | cescens | sp | sp. | nematophilus | nematophilus | rhabdus ne- | escens ne- | sp. | | |
| | | | | | | matophilus | matophilus | | | |
| 42 | S. marce- | Xenorhabdus | P. aerugin- | X. | X. | P. caballi | X. | Photo- | P. lumin- | E. gergoviae |
| | scens and E. | sp | osa | nematophilus | nematophilus | | escens ne- | rhabdus sp. | | |
| | gergoviae | | | | | | matophilus | | | |
| 48 | S. marce- | Xenorhabdus | P. aerugin- | X. | X. | S. marce- | X. | C. freundii | P. lumin- | E. gergoviae |
| | scens and | sp | osa | nematophilus | nematophilus | and | scens ne- | | escens | and Salmon- |
| | Salmonella | | | | and P. | Salmnonella | matophilus | | | ella sp. |
| | sp. | | | | florescens | sp. | | | | |

H. bacteriophora Poinar (Cruiser). Incubation temperature had no apparent effect on the species of bacteria found, but the progression with which the species appeared and disappeared over time was more rapid with higher temperature incubation.

Many bacterial colonies isolated from insects, after nematode exposure of less than 6 h or more than 42 hours were identified as Pasteurella caballi, Salmonella subspecies 1 G and Xanthomonas campestris (Table 2, 3, and 4), these bacteria were also isolated from uninfected insects.

Between 18-24 h incubation, the primary symbiont occurred almost exclusively (Table 2, 3, and 4). Few agar plates during this period grew any other cultures with the exception of H. megidis (M-145) and S. feltiae (27). The primary symbiont from H. megidis (M-145) disappeared rapidly being replaced at 24 h with Serratia proteomaculans. After 24 h of incubation the initial insect contaminants occurred once more. Suppression but not destruction of these organisms may be due to the action of antibiotic compounds produced by the phase I form bacterial symbionts.

Incubation temperature had no effect on the species of bacteria found, but the progression with which the species appeared and disappeared over time was more rapid with higher temperature incubation (Table 2, 3, and 4). At higher temperatures the primary symbiont developed earlier, with the exception of symbionts from S. feltiae (27) and H. megidis (M-145) (Tables 2, 3, and 4). G. mellonella mortality also occurred more rapidly at higher temperatures again with the exception of S. feltiae (27) and H. megidis (M-145) (Table 5).

Only two strains of nematodes carried only the primary symbiont: S. riobrave (355) and H. bacteriophora (Cruiser). Both nematodes came from monoxenic in vitro produced commercial products. Laboratory maintained cultures all associated with other bacterial species. Adsorption of neutral red from MacConkey agar indicated that all primary symbiotic bacteria were phase I form. Bioluminescence was observed from bacterial colonies isolated from H. bacteriophora (Cruiser) and H. marelatus.

Bacteria isolated directly from nematodes revealed several more species as listed in Table 6. It is possible that the insect immune system is able to eliminate such species or that primary symbiotic bacteria proliferating within the insect body produce substances

TABLE 3
Bacterial species identified from Galleria mellonella hemolymph infected with entomopathogenic nematodes at 27° C. over time

| | | | | S. | S. | S. | S. | | H. bac- | |
|---|---|---|---|---|---|---|---|---|---|---|
| | S. | S. riobrave | S. riobrave | carpocapsae | carpocapsae | carpocapsae | carpocapsae | H. | teriophora | H. megidis |
| Time | feltiae (27) | (355) | (Oscar) | (25) | (Kapow) | (Mexican) | (Guardian) | marelatus | (Cruiser) | (M-145) |
| 0 | Salmonella sp., P. caball, and X. campestris isolated from uninfected G. mellaonella | | | | | | | | | |
| 6 | P. caball and | X. camp- | X. camp- | P. caball and | Vibrio sp. | P. caballi | P. caballi | P. caballi | X. camp- | X. camp- |
| | Salmonella | estris | estris And X. | Salmonella | and X. | | and Salmon- | | estris | estris and |
| | sp. | | maltophilia | sp. | nematophilus | | | ella sp. | | Salmonella |
| | | | | | | | | | | sp. |
| 12 | P. caballi | X. camp- | Xenorhabdus | P. caballi | X. | P. caballi | P. caballi | Vibrio sp. | X. camp- | X. camp- |
| | | estris | sp. | | nematophilus | and Vibrio | and Vibrio | | estris | estris |
| | | | | | and P. cab- | sp. | sp. | | | |
| | | | | | alli | | | | | |
| 18 | Vibrio sp. | Xenorhabdus | Xenorhabdus | X. | X. | X. | X. | Photo- | P. lumin- | Photo- |
| | | sp. | sp. | nematophilus | nematophilus | nematophilus | nematophilus | rhabdus sp. | escens | rhabdus sp. |
| 24 | X. boveinii | Xenorhabdus | Xenorhabdus | X. | X. | X. | X. | Photo- | P. lumin- | S. |
| | | sp. | sp. | nematophilus | nematophilus | nematophilus | nematophilus | rhabdus sp. | escens | proteo- |
| | | | | | | | | | | maculans |
| 30 | Vibrio | Xenohabdus | Xenorhabdus | X. | X. | X. | X. | Photo- | P. lumin- | S. |
| | cholerae. | sp | sp. | nematophilus | nematophilus | nematophilus | nematophilus | rhabdus sp. | escens | proteo- |
| | | | | | | | | | | maculans |
| | | | | | | | | | | and Vibrio |
| | | | | | | | | | | met- |
| | | | | | | | | | | schnikovii |
| 36 | S. mar- | Xenorhadbus | Xenorhabdus | X. | X. | X. | X. | Photo- | P. lumin- | E. gergoviae |
| | cescens | sp | sp. | nematophilus | nematophilus | nematophilus | nematophilus | rhabdus sp. | escens | |
| 42 | S. mar- | Xenorhabdus | P. aerugin- | X. | X. | P. caballi | X. | Photo- | P. lumin- | E. gergoviae |
| | cescens and E. | sp | osa | nematophilus | nematophilus | rhadbus sp. | nematophilus | sp. | | |
| | gergoviae | | | | | | | | | |
| 48 | S. mar- | Xenorhabdus | P. aerugin- | X. | X. | S. marescens | X. | C. freundii | P. lumin- | E. gergoviae |
| | cescens and | sp | osa | nematophilus | nematophilus | and | nematophilus | | escens | and Salmon- |
| | Salmonella | | | | and P. | Salmonella | | | | ella sp. |
| | sp. | | | | florescens | sp. | | | | |

TABLE 4
Bacterial species identified from Galleria mellonella hemolymph infected with entomopathogenic nemotodes at 32° C. over time

| | | | | S. | S. | S. | S. | | H. bac- | |
|---|---|---|---|---|---|---|---|---|---|---|
| | S. | S. riobrave | S. riobrave | carpocapsae | carpocapsae | carpocapsae | carpocapsae | H. | teriophora | H megidis |
| Time | feltiae (27) | (355) | (Oscar) | (25) | (Kapow) | (Mexican) | (Guardian) | marelatus | (Cruiser) | (M-145) |
| 0 | Salmonella sp., P. caballi, and X. campestris isolated from uninfected G. mellonella | | | | | | | | | |
| 6 | P. caball and | X. camp- | X. camp- | P. caball and | Vibrio sp | P. caballi | P. caballi | P. caballi | X. camp- | X. camp- |
| | Salmonella | estris | estris | Salmonella | and | | and | | estris | estris |
| | sp | | And | sp. | X. | | Salmonella | | estris | and |
| | | | Xenorhabdus | | nematophilus | | sp. | | | Salomonella |
| | | | | | | | | | | sp. |
| 12 | P. caballi | Xenorhabdus | Xenorhabdus | P. caballi | X. | X. | X. | Vibrio sp. | P. lumin- | X. camp- |
| | | sp | sp. | | nematophilus | nematophilus | nematophilus | | escens | estris |
| 18 | Vibrio sp. | Xenorhabdus | Xenorhabdus | X. | X. | X. | X. | Photo- | P. lumin- | Photo- |
| | | sp. | sp. | nematophilus | nematophilus | nematophilus | nematophilus | rhabdus | escens | proteo- |
| | | | | | | | | sp. | | maculans |
| 24 | X. boveinii | Xenorhabdus | Xenorhabdus | X. | X. | X. | X. | Photo- | P. lumin- | S. |
| | | sp. | sp. | nematophilus | nemtophilus | nematophilus | nematophilus | rhabdus | escens | proteo- |
| | | | | | | | | sp. | | maculans |
| 30 | Vibrio | Xenorhabdus | Xenorhabdus | X. | X. | X. | X. | Photo- | P. lumin- | S. |
| | cholerae. | sp | sp. | nematophilus | nematophilus | nematophilus | nematophilus | rhabdus | escens | proteo- |
| | | | | | | | | sp. | | maculans |
| | | | | | | | | | | and Vibrio |
| | | | | | | | | | | met- |
| | | | | | | | | | | schnikovii |
| 36 | S. mar- | Xenorhabdus | Xenorhabdus | X. | X. | X. | X. | Photo- | P. lumin- | E. gergoviae |
| | cescens | sp | sp. | nematophilus | nematophilus | nematophilus | nematophilus | rhabdus | escens | |
| | | | | | | | | sp. | | |
| 42 | S. mar- | Xenorhabdus | P. aerugin- | X. | X. | P. caballi | X. | Photo- | P. lumin- | E. gergoviae |
| | cescens | sp | osa | nematophilus | nematophilus | | nematophilus | rhabdus | escens | |
| | and E. | | | | | | | sp. | | |
| | gergoviae | | | | | | | | | |
| 48 | S. mar- | Xenorhabdus | P. aerugin- | X. | X. | S. mar- | X. | C. freundii | P. lumin- | E. gergoviae |
| | cescens | sp | osa | nematophilus | nematophilus | cescens and | nematophilus | | escens | and Salmon- |
| | Salmonella | | | | and P. | Salmonella | and | | | ella |
| | sp. | | | | florescens | sp. | Salmonella | | | |
| | | | | | | | sp. | | | |

TABLE 5
Mortality of Galleria mellonella infected with entomopathogenic nematodes at 22, 27, and 32° C., over time

| | | | | | | | | | | H. | |
|---|---|---|---|---|---|---|---|---|---|---|---|
| | Incubation | | S. | S. | S. | S. car- | S. | S. | H. | bacterio- | |
| Time | Temperature | S. feltiae | riobrave | riobrave | carpocapsae | poscapsae | carpocapsae | carpocapsae | mare- | phore | H. megidis |
| (h) | (° C.) | (27) | (355) | (Oscar) | (25) | (Kapow) | (Mexican) | (Guardian) | latus | (Cruiser) | (M-145) |
| 0 | 22 | 0 | 0 | 0 | 0 | 0 | 0 | 0 | 0 | 0 | 0 |
| | 27 | 0 | 0 | 0 | 0 | 0 | 0 | 0 | 0 | 0 | 0 |
| | 32 | 0 | 0 | 0 | 0 | 0 | 0 | 0 | 0 | 0 | 0 |
| 6 | 22 | 0 | 0 | 0 | 0 | 0 | 0 | 0 | 0 | 0 | 0 |
| | 27 | 0 | 0 | 0 | 0 | 0 | 0 | 0 | 0 | 0 | 0 |
| | 32 | 0 | 0 | 0 | 0 | 0 | 0 | 0 | 0 | 0 | 0 |
| 12 | 22 | 60 | 20 | 20 | 40 | 40 | 0 | 20 | 60 | 00 | 20 |
| | 27 | 60 | 60 | 80 | 40 | 40 | 40 | 40 | 40 | 40 | 20 |
| | 32 | 0 | 40 | 40 | 60 | 60 | 60 | 40 | 20 | 40 | 0 |
| 18 | 22 | 80 | 80 | 60 | 80 | 60 | 40 | 40 | 100 | 20 | 60 |
| | 27 | 80 | 80 | 80 | 80 | 80 | 80 | 60 | 80 | 60 | 40 |
| | 32 | 20 | 80 | 100 | 60 | 60 | 60 | 80 | 40 | 80 | 20 |
| 24 | 22 | 100 | 80 | 80 | 100 | 80 | 80 | 80 | 100 | 80 | 100 |
| | 27 | 100 | 100 | 100 | 80 | 100 | 100 | 100 | 100 | 100 | 60 |
| | 32 | 20 | 100 | 100 | 80 | 80 | 100 | 100 | 80 | 100 | 20 |
| 30 | 22 | 100 | 100 | 100 | 100 | 100 | 100 | 100 | 100 | 100 | 100 |
| | 27 | 100 | 100 | 100 | 100 | 100 | 100 | 100 | 100 | 100 | 100 |
| | 32 | 40 | 100 | 100 | 100 | 100 | 100 | 100 | 100 | 100 | 20 |
| 36 | 22 | 100 | 100 | 100 | 100 | 100 | 100 | 100 | 100 | 100 | 100 |
| | 27 | 100 | 100 | 100 | 100 | 100 | 100 | 100 | 100 | 100 | 100 |
| | 32 | 40 | 100 | 100 | 100 | 100 | 100 | 100 | 100 | 100 | 40 |
| 42 | 22 | 100 | 100 | 100 | 100 | 100 | 100 | 100 | 100 | 100 | 100 |
| | 27 | 100 | 100 | 100 | 100 | 100 | 100 | 100 | 100 | 100 | 100 |
| | 32 | 60 | 100 | 100 | 100 | 100 | 100 | 100 | 100 | 100 | 40 |
| 48 | 22 | 100 | 100 | 100 | 100 | 100 | 100 | 100 | 100 | 100 | 100 |
| | 27 | 100 | 100 | 100 | 100 | 100 | 100 | 100 | 100 | 100 | 100 |
| | 32 | 60 | 100 | 100 | 100 | 100 | 100 | 100 | 100 | 100 | 40 |

TABLE 6
Bacteria isolated directly from infective
juvenile entomopathogenic nematodes

| Bacterial | | | | |
|---|---|---|---|---|
| location | Treatment | S. feltiae (27) | S. carpocapsae (Kapow) | S. riobrave (Oscar) |
| On or in | Untreated IJs | Burkholderia cepacia | Enterobacter gergoviae | Burkholderia cepacia |
| nematode | | Flavobacterium indologenes | Pseudomonas sp. | Flavobacterium sp. |
| | | Pseudomonas aeruginosa | Salmonella subspecies 1 G | Serratia marcescens |
| | | Pseudomonas fluorescens type C | Serratia marcescens | Xanthomonas maltophilia |
| | | Salmonella subspecies 1 G | Xenorhabdus nematophilus | Xenorhabdus sp. |
| | | Xenorhabdus bovienii | | |
| In nematode | Surface sterilized | Burkholderia cepacia | Enterobacter gergoviae | Burkbolderia cepacia |
| | IJs | Flavobacterium indologenes | Pseudomonas sp. | Flavobacterium sp. |
| | | Pseudomonas aeruginosa | Salmonella subspecies 1 G | Serratia marcescens |
| | | Pseudomonas fluorescens type C | Serratia marcescens | Xanthomonas maltophilia |
| | | Salmonella subspecies 1 G | Xenorhabdus nematophilus | Xenorhabdus sp. |
| | | Xenorhabdus bovienii | | |

that kill the bacteria. The present invention therefore contemplates the use of mixtures of these bacteria isolated from insect hemolymph infected with nematodes for more effective control of insects. Specifically, the present invention contemplates the use of mixtures of one or more of the bacteria Burkholderia cepacia, Flavobacterium spp. (such as F. indologenes), Pseudomonas spp. (such as P. aeruginosa and P. fluorescens type C), Salmonella Subspecies 1G, Enterobacter gergoviae, Serratia marcescens, Xanthomonas maltophilia with Xenorhabdus spp. (such as X. bovienii, X. luninescens, and X. nematophilus) and/or Photorhabdus spp. (such as P. luminescens).

The correlation between bacterial motility, culture growth curve, and insect mortality was established by the following examples. Three-sided Erlenmeyer culture flasks were autoclaved for 20 mins, each with 150 ml of LB broth (Miller) (1% Tryptone (Difco), 0.5% yeast extract (Difco), 1% NaCl). Two screw-capped culture tubes, each containing 5 ml LB were also autoclaved for 20 mins, cooled, inoculated with 1 loop Xenorhabdus nematophilus Im-1, and incubated at room temperature for approximately 24 hrs. Each of the Erlenmeyer flasks were then inoculated with the 5 ml culture from the screw-capped 24 hr. cultures and then incubated at room temperature. One of the two flasks was shaken at 90 rpm on a rotary shaker and the other was left undisturbed as a stationary culture. The turbidity of each culture was measured in Klett units (approximately O.D.₆₆₀) for 15 samples taken approximately every 4 hrs for a total of 98 hours of time in culture. Samples were also taken to determine the percentage of the population that exhibited motility. Cultures were shaken gently prior to sampling to increase the likelihood that the sample was of uniform character. Percentage motility was estimated visually by examining diluted culture samples in a hanging drop with the aid of a light microscope and an oil immersion objective lens.

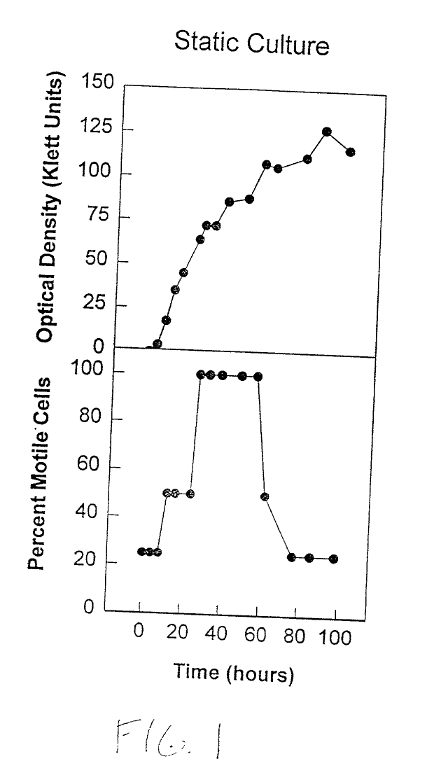

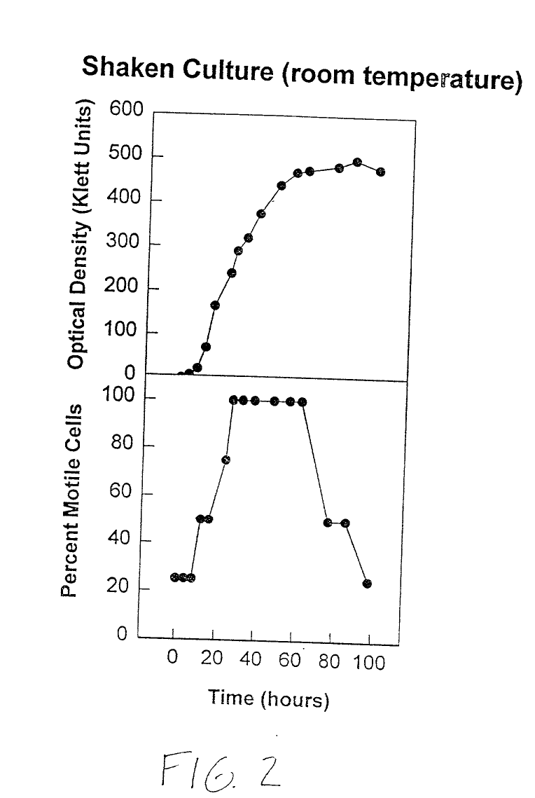

As shown in FIGS. 1 and 2, the growth curves of the shaken and static cultures were quite similar, both reaching maximum phases at about 86 hours of culture and 100% motility between about 27 and 56 hours of culture. Thus the highest motility occurs at approximately the mid log phase of bacterial growth prior to their entering stationary phase. The primary difference between the two cultures is in the extent of the turbidity. The static culture reached 128 Klett units and the shaken culture reached 500 Klett units in 86 hours. Because the turbidity of the culture represents the number of bacterial cells per unit volume, the shaken culture contains more than four times as many bacterial cells as the static culture, allowing a four-fold dilution while effectively maintaining the same number of cells as the static culture.

To test the efficacy of the motile bacteria on insects, a 26 hr culture of X. nematophilus ATCC 55585 was diluted 80 to 1 with deep well water, making 10 gals. With culture and air temperature at about 79° F., and using a 12 volt electric pump (pressure about 10-15 pounds), 20 active fire ant (Solenopsis invicta) mounds were sprayed with a stream of diluted culture until the mounds were flattened. The average application was about one-half gallon of diluted culture. At approximately 48 hours, ant mounds were examined by digging to provoke a response. At 96 hours, all mounds were void of ants except for one in which fewer than ten ants responded to the digging and they appeared dibilitated. One mound had relocated a few feet away.

On the same date and at approximately the same time, the same procedure and equipment was used to apply two other diluted cultures of bacteria harvested from nematodes to twelve fire ant mounds of each strain except that the temperature of the water in which the bacteria was diluted was slightly cooler than ambient temperature. The first of the two bacterial cultures was harvested from S. carpocapsae (Mex) and the second was harvested from S. scapterisci; no attempt was made to identify the specific bacteria cultured such that it is expected that the bacteria was a mix of the bacteria harvested from the nematodes as described above. About half the ant mounds were devoid of any ant activity at 72 hrs in the case of the bacterial culture harvested from S. carpocapsae (Mex) and the bacterial cultures harvested from S. scapterisci showed this same reduction at about 48 hrs. These same cultures killed nine mounds (with reduced activity and number of ants in the other three) at 120 hours and eight mounds (with the other four still showing activity), respectively.

Similar field tests have been conducted with a different genus of bacteria, Serratia marcescens, isolated from the nematode Heterorhabditis bacteriophora (Oswego) with the final result (over about three weeks) that 24 of 25 mounds of fire ants were killed. Similar results were obtained in tests conducted at the same time with bacteria isolated from Steinernema kushidai and S. poinarii.

As noted above, the diluted bacterial culture is applied to the ant mound by spraying. The mode of application of the bacteria and the rate of application depends upon the nature of the insect to be controlled and such factors as environmental conditions, the motility of the bacteria, the liquid in which the bacteria are suspended, the proportion of liquid to bactera, and many other factors that will be known to those skilled in the art who have the benefit of this disclosure. The number of bacterial cells is determined relatively easily by appropriate dilution and where larger amounts of bacteria are needed to control the insect pest, an adequate volume of the dilute culture can even be poured onto, for instance, ant or termite mounds. Broadcast spraying may be appropriate for controlling, for instance, chinch bug infestations in grass or gypsy moth infestations in deciduous trees (if the insects are found to be susceptible under those conditions).

Although not a requirement of the present invention, suitable adjuvants may also be added to the diluted bacterial culture for application to the insect population. Although data indicates variable results, diatomaceous earth has long been considered a suitable adjuvant for dilute solutions of insect control agents for such insects as fire ants (on the assumption that the diatomaceous earth wounds the ants). The present invention also contemplates the addition of various commercially-available detergents and wetting agents to dilute bacterial cultures, with and without diatomaceous earth, as an adjuvant.

Field tests have also been conducted with bacteria isolated from S. carpocapsae ALL, S. carpocapsae Kapow, and S. carpocapsae ATCC 19601. The bacteria cultured from each of these nematodes showed varying degrees of insecticidal effect (mortality) against bag worms, fleas, ticks, and termites in field testing. The results of more controlled tests with several bacterial species correlate with the results of these field tests. Specifically, X. poinarii, X. nematophilus, and P. luminescens were cultured in nutrient broth for 56 hr at 24° C., at which point the bacteria were growing exponentially and were highly motile. Experimental arenas were constructed in 15 cm petri dishes. 50 fire ants or termites were placed in each arena with 40 ml sandy loam soil. Three replicate dishes were run for each combination of bacterial strain and insect species. Termites were offered wooden tongue depressors as a cellulose source while ants received two late instar G. mellonellla larvae as a food source. Each petri dish received about 360 million bacterial cells per dish of 50 insects. The bacteria were pipetted directly into the areas in 6 ml of nutrient broth. Control treatments consisted of plates containing termites or ants treated with 6 ml of sterile nutrient broth. The dishes were evaluated at 24, 48, 72, 96, 144, and 168 hours and percent mortalities were estimated for each replicate plate at each evaluation point. The bacteria/strains tested included:

Xenorhabdus nematophilus Im1 isolated from Steinernema carpocapsae (ALL)

Xenorhabdus spp. (#32) isolated from Steinernema scapterisci

Xenorhabdus poinarii (#106) isolated from Steinernema glaseri (#106)

Xenorhabdus sp. (K) isolated from Steinernema kushidai

Xenorhabdus sp. (A) isolated from Steinernema anomali

Xenorhabdus sp. (Red and Blue isolates) Isolated from field collected boll weevil

Photorhabdus luminescens (#29) isolated from Heterorhabditis bacteriophora (HP88)

Photorhabdus luminescens (w/p) isolated from Heterorhabditis bacteriophora (Rich C)

Phtorhabdus sp. isolated from Heterorhabditis hawaienisi

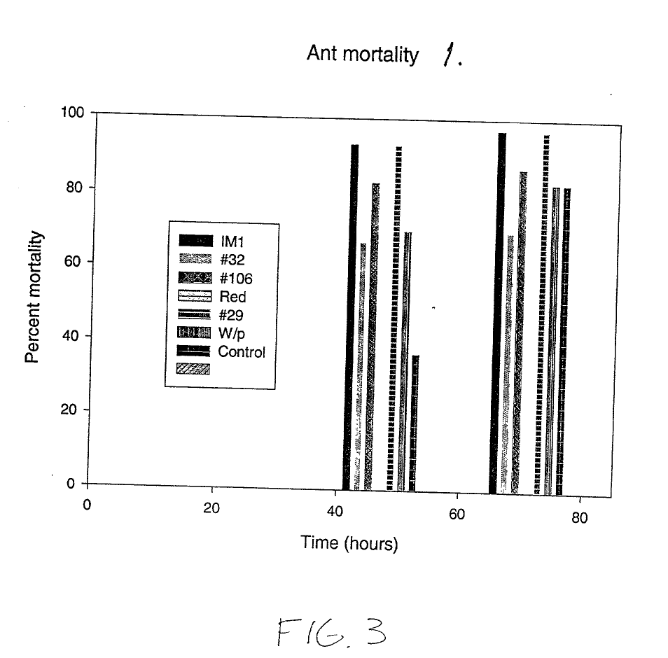

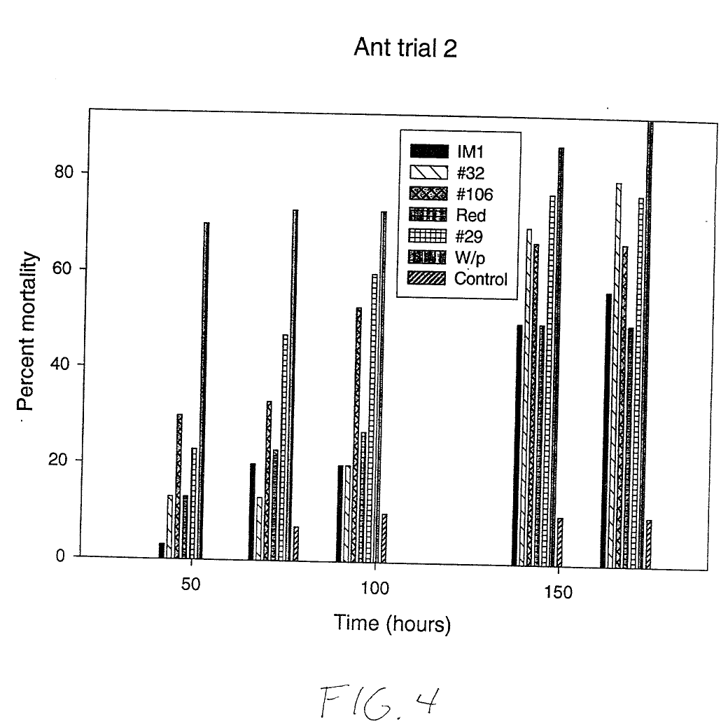

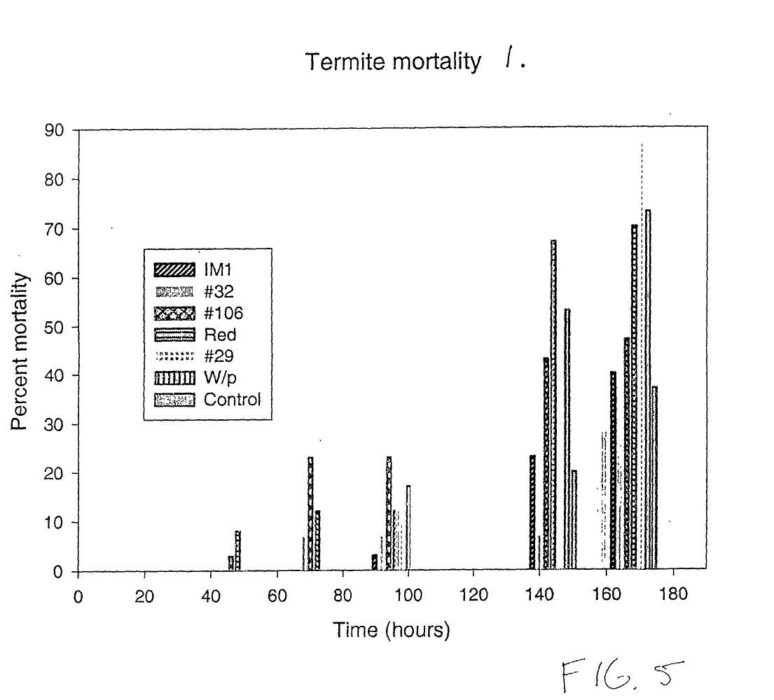

Although fungal contamination problems affected some of the plates, as shown in FIGS. 3 and 4, ant mortalities in two separate trials ranged from none at 24 hours to as much as 100% ant mortality at 48 hours. As shown in FIG. 5, termite mortality was generally lower and slower, ranging from no mortality at 24 hours to 100% mortality on some plates at 168 hours. Dead insects were collected periodically and the cadavers were surface sterilized (0.1% thimerosal solution for 15 mins., then rinsed in deionized water twice) and the insect hemocoel streaked on TSA plates. Resulting bacterial colonies were identified using the BIOLOG® bacterial identification system using an immunological test using a polyclonal antibody specific to the surface antigens of Xenorhabdus and Photorhabdus species. Using this technique, P. luminescens (w/p) was successfully re-isolated from the termite hemocoel.

The foregoing description of the preferred embodiment of the invention has been presented for purposes of illustration and description. It is not intended to be exhaustive or to limit the invention to the precise form disclosed, and many modifications and variations are possible in light of the above teaching without deviating from the spirit and the scope of the invention. The embodiment described is selected to best explain the principles of the invention and its practical application to thereby enable others skilled in the art to best utilize the invention in various embodiments and with various modifications as suited to the particular purpose contemplated. It is intended that the scope of the invention be defined by the claims appended hereto.

What is claimed is:

1. A method of maintaining the virulence of bacteria harvested from nematodes comprising the steps of:
maintaining a library of nematodes selected from the families Steinernematidae and Heterorhabditidae in a suitable culture media under conditions conducive to the health of the nematodes;
maintaining separate populations of the bacteria species harvested from the nematodes in suitable culture media under conditions conducive to the reproduction of the bacteria;
periodically inoculating one of the bacterial species maintained in separate populations into a suitable nematode host selected from the library of nematodes;
incubating the inoculated nematode host under conditions conducive to the reproduction of the inoculated bacterial species; and
re-isolating the inoculated bacterial species from the nematode host.

2. The method of claim 1 additionally comprising identifying the bacterial species re-isolated from the inoculated nematode.

3. An insecticidally-active composition comprising a mixture of one or more bacterial species harvested from nematodes of the families Steinernematidae or Heterorhabditidae, said nematodes being maintained in a suitable storage medium, and water, said bacterial species being selected from the genera Xenorhabdus, Photorhabdus, Pseudomonas, Burkholderia, Flavobacterium, Enterobacter, Serratia, Xanthomonas, and Vibrio.

4. The insecticidally-active composition of claim 3 additionally comprising an adjuvant.

5. The insecticidally-active composition of claim 3 wherein said bacterial species are harvested directly from the nematodes rather than from the insect host.

6. The insecticidally-active composition of claim 5 wherein said bacterial species are harvested from the nematodes while the nematodes are at the infective juvenile stage.

7. The insecticidally-active composition of claim 3 wherein the bacterial species comprising said composition are harvested from the nematodes and cultured as separate bacterial populations and the one or more bacterial species included in said composition is harvested from the culture during the exponential growth phase of the culture.

8. The insecticidally-active composition of claim 3 wherein the bacterial species comprising said composition are harvested from the nematodes and cultured as separate bacterial populations and the one or more bacterial species included in said composition is harvested from the culture at a time when the bacterial species is highly motile.
